(19) United States
(12) Patent Application Publication
(10) Pub. No.: US 2004/0228845 A1
(43) Pub. Date: Nov. 18, 2004

(54) Methods of using CD8+/TCR- facilitating cells (FC) for the engraftment of purified hematopoietic stem cells (HSC)

(76) Inventor: Suzanne T. Ildstad, Prospect, KY (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/438,264

(22) Filed: May 14, 2003

(51) Int. Cl. A61K 45/00; C12N 5/08

(52) U.S. Cl. 424/93.7; 435/372

(57) ABSTRACT

The present invention relates to the identification and use of facilitating cells that are critical for engraftment of purified “hematopoietic stem cells” (HSC), and more specifically this invention relates to two cell populations of CD8⁺ cells, that is, CD8⁺/TCR⁻ “facilitating cells” (FC) which are critical to “hematopoietic stem cells” (HSC) survival and self-renewal, and CD8⁺/TCR⁺ cells which enhance the level of donor engraftment but do not promote long-term, durable engraftment. These two cell populations may have a wide range of applications, including but not limited to, hematopoietic reconstitution by bone marrow transplantation for the treatment of cancers, anemias, autoimmunity, immunodeficiency, viral infections and metabolic disorders as well as facilitation of solid organ, tissue and cellular transplantation.

### CROSS-REFERENCE TO RELATED APPLICATIONS

This application is a Section 371 filing of PCT/US01/45312, filed Nov. 14, 2001, which claims priority to U.S. Provisional Application Serial No. 60/248,895 filed Nov. 14, 2000, the disclosures of which are incorporated herein by reference.

### CONTRACTUAL ORIGIN OF THE INVENTION

This research was supported in part by the National Institutes of Health, grant DK43901-07. The government has certain rights in the invention.

### BACKGROUND OF THE INVENTION

1. Field of the Invention

The present invention relates to the identification and use of facilitating cells that are critical for engraftment of purified “hematopoietic stem cells” (HSC), and more specifically this invention relates to two cell populations of CD8⁺ cells, that is, CD8⁺/TCR⁻ “facilitating cells” (FC) which are critical to “hematopoietic stem cells” (HSC) survival and self-renewal, and CD8⁺/TCR⁺ cells which enhance the level of donor engraftment but do not promote long-term, durable engraftment.

2. Description of the State of Art

The transfer of living cells, tissues, or organs from a donor to a recipient, with the intention of maintaining the functional integrity of the transplanted material in the recipient defines transplantation. Transplants are categorized by site and genetic relationship between the donor and recipient. An autograft is the transfer of one's own tissue from one location to another; a syngeneic graft (isograft) is a graft between identical twins; an allogeneic graft (homograft) is a graft between genetically dissimilar members of the same species; and a xenogeneic graft (heterograft) is a transplant between members of different species.

A major goal in solid organ transplantation is the permanent engraftment of the donor organ without a graft rejection immune response generated by the recipient, while preserving the immunocompetence of the recipient against other foreign antigens. Typically, in order to prevent host rejection responses, nonspecific immunosuppressive agents such as cyclosporine, methotrexate, steroids and FK506 are used. These agents must be administered on a daily basis and if stopped, graft rejection usually results. However, a major problem in using nonspecific immunosuppressive agents is that they function by suppressing all aspects of the immune response, thereby greatly increasing a recipient's susceptibility to opportunistic infections, rate of malignancy, and end-organ toxicity. The side effects associated with the use of these drugs include opportunistic infection, an increased rate of malignancy, and end-organ toxicity (Dunn, D. L., Crit. Care Clin., 6:955 (1990)). Although immunosuppression prevents acute rejection, chronic rejection remains the primary cause of late graft loss (Nagano, H., et al., Am. J. Med. Sci., 313:305-309 (1997)).

For every organ, there is a fixed rate of graft loss per annum. The five-year graft survival for kidney transplants is 74% (Terasaki, P. I., et al., UCLA Tissue Typing Laboratory (1992)). Only 69% of pancreatic grafts, 68% of cardiac transplants and 43% of pulmonary transplants function 5 years after transplantation (Opelz, G., Transplant Proc., 31:31S-33S (1999)).

The only known clinical condition in which complete systemic donor-specific transplantation tolerance occurs is when chimerism is created through bone marrow transplantation. (Qin, et al., J. Exp. Med., 169:779 (1989); Sykes, et al., Immunol. Today, 9:23-27 (1988); and Sharabi, et al., J. Exp. Med., 169:493-502 (1989)). This has been achieved in neonatal and adult animal models as well as in humans by total lymphoid or body irradiation of a recipient followed by bone marrow transplantation with donor cells. The success rate of allogenic bone marrow transplantation is, in large part, dependent on the ability to closely match the “major histocompatability complex” (MHC) of the donor cells with that of the recipient cells to minimize the antigenic differences between the donor and the recipient, thereby reducing the frequency of host-versus-graft responses and “graft-versus-host disease” (GVHD). In fact, MHC matching is essential, only a one or two antigen mismatch is acceptable because GVHD is very severe in cases of greater disparities.

The MHC is a cluster of closely linked genetic loci encoding three different classes (class I, class II, and class III) of glycoproteins expressed on the surface of both donor and host cells that are the major targets of transplantation rejection immune responses. The MHC is divided into a series of regions or subregions and each region contains multiple loci. An MHC is present in all vertebrates, and the mouse MHC (commonly referred to as H-2 complex) and the human MHC (commonly referred to as the Human Leukocyte Antigen or HLA) are the best characterized.

The development of safe methods to achieve mixed allogeneic chimerism to induce donor-specific tolerance across MHC barriers remains a major goal. Two barriers associated with bone marrow transplantation BMT have limited its application to clinical transplantation: (1) graft-versus-host disease (GVHD) and (2) failure of engraftment. T-cell depletion (TCD) of donor marrow can eliminate GVHD but is associated with a significant increase in graft failure. Consequently, it was hypothesized that T-cells are required for durable engraftment of allogeneic hemalopoietic stem cells (HSC). Although highly purified HSC engraft readily in syngeneic and MHC-congenic recipients, they do not engraft as readily in MHC-disparate recipients. The addition of CD8⁺/TCR graft facilitating cells (FC) overcomes this limitation in mouse. In the rat, depletion of CD8⁺, CD3⁺ or CD5⁺ cells from the donor marrow is associated with a significant increase in failure of engraftment.

The role of MHC was first identified for its effects on tumor or skin transplantation and immune responsiveness. Different loci of the MHC encode two general types of antigens which are class I and class II antigens. In the mouse, the MHC consists of 8 genetic loci: Class I is comprised of K and D, class II is comprised of I-A and /or I-E. The class II molecules are each heterodimers, comprised of I-Aα and I-Aβ and/or I-Eα and I-Eβ. The major function of the MHC molecule is immune recognition by the binding of peptides and the interaction with T-cells, usually via the αβ T-cell receptor. It was shown that the MHC molecules influence graft rejection mediated by T cells (Curr. Opin. Immunol., 3:715 (1991), as well as by NK cells (Annu. Rev. Immunol., 10:189 (1992); J. Exp. Med., 168:1469 (1988); Science, 246:666 (1989). The induction of donor-specific tolerance by HSC chimerism overcomes the requirement for chronic immunosuppression. (Ildstad, S. T., et al., Nature, 307:168-170, (1984), Sykes, M., et al., Immunology Today, 9:23-27 (1998), Spitzer, T. R., et al., Transplantation, 68:480-484, (1999)). Moreover, bone marrow chimerism also prevents chronic rejection (Colson, Y., et al., Transplantation, 60:971-980 (1995); and Gammie, J. S., et al., In Press Circulation (1998)). The association between chimerism and tolerance has been demonstrated in numerous animal models including rodents (Ildstad, S. T., et al. Nature, 307:168-170, (1984); and Billingham, R. E., et al., Nature, 172:606 (1953)), large animals, primates and humans (Knobler, H. Y., et al., Transplantation, 40:223-225 (1985); Sayegh, M. H., et al., Annals of Internal Medicine, 114:954-955 (1991)).

T cells can be divided into two populations: αβ-TCR⁺ T cells and γδ-TCR⁺ T cells. αβ-T cell receptor (TCR)⁺ T cells are the predominant circulating population and can be subdivided into cells expressing CD4⁺ or CD8⁺ antigens. γδ-TCR⁺ T cells represent approximately 2% of peripheral T cells and are predominantly CD3⁺ but CD4⁻/CD8⁻. The role of αβ-TCR⁺ T cells in the pathophysiology of acute GVHD is supported by a number of studies. The role of γδ-TCR⁺ T cells as effector cells for GVHD has been debated. Data from recently developed transgenic murine models indicate that a clonal population of γδ-TCR⁺ T cells are capable of inducing acute GVHD, as well as mediating graft rejection. Blocking the ability of the TCR to bind to the host MHC through the use of peptides that target the MHC has led to reduction in GVHD. Elucidating the participation of αβ and γδ-TCR⁺ subsets in GVHD is a necessary step in the goal of removing the T cells responsible for GVHD, and on evaluating the influence of the cellular subsets on engraftment.

Highly purified hematopoietic stem cells (HSC) engraft readily in syngeneic and MHC congenic recipients while engraftment is significantly impaired in MHC-disparate allogeneic recipients. The addition of CD8⁺ graft facilitating cells (FC) restores engraftment-potential of highly purified HSC in allogeneic recipients in vivo (Sharkas, Martin, Weissman, Ildstad JEM; Kaufman, et al. Blood, 84:2436-2446 (1994)). The precise phenotype source, and biological role of CD8⁺ FC has remained controversial (Martin, Immunity). As few as 10,000 CD8⁺/TCR⁺/CD3ε bone marrow-derived FC have been demonstrated to enable durable engraftment of HSC in fully ablated (950 cGy TBI) mice (Kaufman, Blood; Colson, Nat. MED). CD8⁺/TCR⁺ lymphnode-derived FC are essential to engraftment of marrow in MHC disparate recipients conditioned with 800 cGy TBI (Martin, JEM). In mice conditioned with 800 cGy TBI, CD8⁺/TCR⁻ bone marrow-derived FC facilitated engraftment, but CD8^{total} (TCR⁺ plus TCR⁻) cells combined mediated the most potent engraftment-enhancing biologic effect (Weissman, Immunity). Because the majority of CD8⁺/TCR⁻ cells in marrow are CD3ε⁻, it was concluded that the biologic activity resided in this cellular fraction rather than the more infrequent CD3ε⁺ population (Weissman Immunity). In the present experiments we have resolved this controversy and demonstrated that CD8⁺/TCR⁺/CD3ε⁺ FC are critical to durable HSC engraftment while CD8⁺/TCR⁻/CD3ε⁺ T cells are only supplemental. Moreover, TCR βδ KO mice produce FC, while CD3ε transgenic (TG) mice do not, suggesting a lymphoid-derived non T cell lineage for CD8⁺/TCR⁻ FC.

Two populations of CD8⁺ cells in bone marrow have been described to facilitate engraftment of highly purified hematopoietic stem cells (HSC) in MHC-disparate allogeneic recipients. CD8⁺/TCR⁻ facilitating cells (FC) facilitate durable engraftment of HSC without causing GVHD, while CD8⁺/TCR⁻ FC plus CD8⁺/TCR⁺ cells may also facilitate. CD8⁺/TCR⁺ cells alone are not sufficient to support long-term graft survival. Without FC, HSC prolong survival, but do not promote sustained engraftment.

Bone marrow transplantation (BMT) has the potential to treat a number of genetic disorders, including hemoglobinopathies (sickle cell disease, thalassemia), soluble enzyme deficiencies, and autoimmune disorders. The morbidity and mortality associated with transplantation of unmodified marrow has prevented the widespread application of this approach. Conventional T cell depletion prevents graft versus host disease but is associated with an unacceptably high rate of graft failure. A better understanding of the biology of engraftment of HSC will allow approaches to graft engineering to optimize engraftment and avoid the risks associated with BMT.

Therefore, there remains a need for an optimization of engraftment procedures.

### SUMMARY OF THE INVENTION

Accordingly, one aspect of this invention provides methods for conditioning a recipient for bone marrow transplantation which eliminates the need for nonspecific immunosuppressive agents and/or lethal irradiation. More specifically, one method of this invention comprises introducing CD8⁺/TCR⁻ facilitating cells and purified hematopoietic stem cells into a recipient lacking T-cells.

This invention further identifies which cells in the host recipient microenvironment influence alloresistance to engraftment.

Another aspect of the invention is to deplete or preferably eliminate those cells in the host environment which influence alloresistance to engraftment thereby conditioning the recipient for engraftment.

This invention provides method for treating a variety of diseases and disorders with minimal morbidity.

Additional advantages and novel features of this invention shall be set forth in part in the description and examples that follow, and in part will become apparent to those skilled in the art upon examination of the following or may be learned by the practice of the invention. The advantages of the invention may be realized and attained by means of the instrumentalities and in combinations particularly pointed out in the appended claims.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

In the Drawings:

FIG. 1 illustrates T-cell depletion of rat bone marrow.

FIGS. 2A, 2B, and 2C illustrate the detection of facilitating cells.

FIG. 3 illustrates the analysis of CD8⁺/TCR⁻ FC for expression of CD11a and CD11c.

FIG. 4 is a table illustrating the assessment of BVHD after bone marrow transplantation.

FIGS. 5A-E illustrate a histologic assessment of GVHD.

FIG. 6 illustrates the survival of heterotopic cardiac allografts in mixed allogeneic chimeras (ACI→WF).

### DETAILED DESCRIPTION OF THE PREFERRED EMBODIMENT

The present invention is based on the hypothesis that CD8⁺/TCR⁻ FC are critical to HSC survival and self-renewal, while CD8⁺/TCR⁺ conventional T-cells are supplemental and do not promote long-term, durable engraftment. Further, donors lacking TCR⁻β/δ may still produce facilitating cells (FC). And, that depletion of αβ- and γδ-TCR⁺ T cells will not affect the engraftment-potential of the rat bone marrow cells, since their depletion should leave the FC population intact. In the present invention a bone marrow transplant (BMT) was engineered in which the αβ- and γδ-TCR⁺ T cells were depleted from donor marrow. The role of each cell population in engraftment and graft-versus-host disease (GVHD) was subsequently evaluated. Depletion of both αβ- and γδ-TCR⁺ T cells from donor marrow allowed durable engraftment, but completely avoided GVHD. The resulting chimeric animals exhibited stable mixed allogeneic chimerism and donor-specific tolerance to cardiac grafts for one year. These data are consistent with the hypothesis that FC, although CD3⁺, are not “conventional” T cells, because they do not express T cell receptor (TCR). The present invention indicates that αβ- or γδ-TCR⁺ T cells are sufficient to cause GVHD, and that the presence of either αβ- or γδ-TCR⁺ T cells in the donor marrow inoculum affects the level of donor chimerism. These data confirm that neither αβ- nor γδ-TCR⁺ T cells are required for durable HSC engraftment in MHC-disparate recipients, but that both contribute to GVHD as well as to influence the level of donor chimerism.

GVHD currently limits the clinical application of BMT for the induction of donor specific tolerance. Strategies to T cell deplete the bone marrow of GVHD-producing cells prevents GVHD, but is associated with a significant increase in failure of engraftment. The rat is a superior model to study GVHD and TCD graft failure because it is more prone to GVHD as well as failure of engraftment compared to the mouse. Depletion of T cells from the rat marrow using anti-CD5, anti-CD8, or anti-CD3 mAb decreases the incidence of GVHD but also results in increased occurrence of graft failure after allogeneic bone marrow transplant. A cell population in mouse bone marrow (CD8⁺/CD3⁺/CD5⁺/TCR⁻), separate from the HSC, that facilitates engraftment of purified allogeneic HSC without causing GVHD. Because the FC shares some cell surface molecules with T cells, it is not known whether the T cell depletion-related graft failure is due to the depletion of facilitating cell populations or conventional T cell populations. Recent studies suggest that the CD8⁺/TCR⁺ and CD8⁺/TCW subpopulations of marrow facilitate the engraftment of allogeneic HSC, but that the CD8⁺/TCR⁻ cells are the most potent effector cells and have the added advantage that they do not cause GVHD. Moreover, a facilitating role for CD8⁺ lymphnode lymphocytes and γδ T cells has also been reported. In continuing studies in the mouse, purified FC allow physiologic numbers of HSC to engraft in allogeneic recipients, while purified T cells do not. However, purified T cells enhance engraftment in partially conditioned mouse recipients if FC are present.

In the present invention, it has been determined whether and how αβ- and γδ-TCR⁺ T cells contribute to engraftment of HSC. It was hypothesized that in the previous studies the TCD strategy was removing FC as well as T cells, resulting in graft failure, and that removal of T cells (αβ- and γδ TCR⁺ T cells) with sparing of FC would not result in impaired engraftment. Virtually all recipients of marrow depleted of either αβ- or γδ-TCR⁺ T cells engrafted. Similarly, all the recipients transplanted with donor marrow aggressively depleted of αβ- and γδ-TCR⁺ T cells engrafted and exhibited stable mixed HSC chimerism. These data therefore demonstrate that depletion of αβ and γδ-TCR⁺ T cells allows engraftment of allogeneic HSC.

It is important to note that the CD3⁺/CD8⁺/TCR⁻ FC cell population remained in the donor cell inoculum after αβ- and γδ-TCR⁺ T cell depletion. The ontogeny of FC and lineage derivation have not yet been defined. The FC population is separate from the conventional T cell population when analyzed by flow cytometry in that CD8 and CD3 expression are less intense than that for CD8⁺ T cells. Moreover, the FC population is predominantly CD11c positive, suggesting a possible dendritic cell ontogeny. Taken together, these data therefore indirectly support the existence of a facilitating cell population, separate from conventional T cells, in rat bone marrow. Because there is no strategy currently available to purify rat HSC, we are unable to sort only FC plus HSC and co-administer them in purified form to ablated rat recipients.

Although TCD did not influence engraftment, the percentage of donor chimerism was significantly influenced by the composition of the marrow inoculum. The role of γδ-TCR⁺ T cells in influencing engraftment has been debated. Recipients of marrow depleted of both αβ plus γδ-TCR⁺ T cells repopulated with significantly lower levels of mixed chimerism compared to those administered marrow containing αβ-TCR⁺ T cells (46.3%±32.8% and 92.3%±9.2%, respectively; p<0.05). Moreover, recipients of marrow containing γδ-TCR⁺ T cells also exhibited higher levels of donor chimerism. These data suggest that while conventional T cells are not required for engraftment of the HSC, they do influence the level of chimerism established. These data resolve the apparent dichotomy between the report of facilitating cells and others in which lymph node CD8⁺ T cells were demonstrated to enhance the level of chimerism, since FC were present in the marrow used in his studies. Moreover, while αβ- or γδ-TCR⁺ T, cells are not required for durable engraftment, they do significantly influence level of chimerism.

Also evaluated was the role of T cell subsets in mediating GVHD and influencing engraftment-potential. It has been debated whether γδ-TCR⁺ T cells can mediate GVHD. One study showed that the γδ-TCR⁺ T cell does not play a role in GVHD in mice, while another showed that cells co-expressing γδ-TCR⁺ and natural killer (NK)1.1⁺ play a role in the pathogenesis of acute GVHD. However, the mouse is an inferior model for these studies because it is much more resistant to GVHD. The rat is more prone to GVHD and is therefore a superior model. Although the depletion of γδ-TCR⁺ T cells alone did not significantly affect the development of GVHD, the depletion of γδ-TCR⁺ T cells in addition to αβ-TCR⁺ T cells completely avoids GVHD. None of these animals exhibited clinical signs of GVHD, while only minimal signs of GVHD (grade 1) were detected histologically. These data clearly demonstrate that although the αβ-TCR⁺ T cells play a dominant role, γδ-TCR⁺ T cells also contribute in an independent fashion to GVHD. It has been previously demonstrated that depletion of αβ-TCR⁺ T cells from donor marrow decreased the occurrence of GVHD while preserving engraftment in rats. The results reported here are consistent with those results, indicating that αβ-TCR⁺ T cells are important in mediating GVHD in rats. Although all the recipients reconstituted with marrow depleted of αβ-TCR⁺ T cells but containing γδ-TCR⁺ T cells exhibited clinical or histological signs of GVHD, the severity of the disease was decreased compared with recipients of marrow containing αβ-TCR⁺ T cells. These data confirm that, while αβ-TCR⁺ T cells mediate GVHD in the rat, γδ-TCR⁺ T cells are also capable of inducing GVHD independent of αβ-TCR⁺ T cells.

The selective depletion of marrow of either αβ- or γδ-TCR⁺ T cell subsets allowed us to evaluate whether the specific cell types resulted in a differential occurrence of GVHD. Recipients of marrow depleted of only γδ-TCR⁺ T cells developed moderate to severe GVHD relatively early after BMT (30 days post-transplantation), primarily affecting the skin and tongue. Depletion of only αβ-TCR⁺ T cells resulted in more mild GVHD affecting primarily in the liver and small intestine at 150 and 220 days post-BMT. However, when both αβ- and γδ TCR⁺ T cells were depleted, severe GVHD was prevented. These data suggest that αβ-γδ-TCR⁺ T cell subsets target different tissues and mediate their affect at different times. αβ-TCR⁺ T cells result in GVHD histologically by destruction of skin, tongue early post-BMT; γδ-TCR⁺ T cells have the capability of causing GVHD target in liver and small intestine late post-BMT.

The induction of tolerance has the potential to overcome the two major problems that currently limit organ transplantation: chronic rejection and the complications associated with immunosuppressive therapy. Mixed allogeneic chimerism induces donor-specific transplantation tolerance to solid organ grafts. It has been debated whether donor T cells must be present in the marrow inoculum for tolerance to be achieved. Such T cells were hypothesized to “balance” the recipient T cells. It is hypothesized that mixed chimerism induces deletional tolerance and that donor T cells are not required for tolerance to be induced. The mixed chimeras generated using marrow depleted of both αβ- and γδ-TCR⁺ T cells exhibit donor-specific tolerance to solid organ grafts. Mixed chimeras accept donor-specific cardiac grafts (MST>375 days) without evidence of chronic rejection while third-party cardiac grafts are rejected as rapidly as untreated control rats. These data therefore confirm that mature donor T cells are not required to induce tolerance through mixed HSC chimerism. The present invention results culminates in the hypothesis that the engraftment of the donor pluripotent HSC in the form of mixed chimerism allows deletional tolerance to occur as newly produced host- and donor-derived lymphocytes are produced. The presence of donor-derived dendritic cells in the thymus of mixed chimeras provides a potent deleting ligand for any donor-reactive T cells of host or donor origin, resulting in a robust form of tolerance.

In summary, the present invention demonstrates that αβ- and γδ-TCR⁺ T cells affect the level of donor chimerism but not engraftment, since depletion of αβ- and γδ-TCR⁺ T cells from the donor bone marrow retains engraftment-potential yet avoids GVHD, suggesting that an FC population is present functionally as well as phenotypically in rat bone marrow. Moreover, both αβ- and γδ-TCR⁺ T cells mediate GVHD. However, αβ-TCR⁺ T cells mediate more severe GVHD with a more rapid onset than the GVHD mediated by γδ-TCR⁺ T cells. Strategies to engineer a BMT to remove GVHD-producing cells but retain facilitating cells may allow the clinical application of BMT to induce tolerance to solid organ and cellular grafts to become a reality.

Thus, the present invention relates to a composition comprising two cell populations of CD8⁺ cells, that is, CD8⁺/TCR⁻ “facilitating cells” (FC) which are critical to “hematopoietic stem cells” (HSC) survival and self-renewal, and CD8⁺/TCR⁺ cells which enhance the level of donor engraftment but do not promote long-term, durable engraftment.

Generally, purified or partially purified FC facilitate engraftment of stem cells which are MHC-specific to the FC so as to provide superior survival of the chimeric immune system. The stem cells and FC preferably come from a common donor or genetically identical donors. However, if the donor is of a species or a strain of a species which possesses a universal facilitatory cell, the stem cells need not be MHC-specific to the facilitatory cell. By purifying the FC separately, either by positive selection, negative selection, or a combination of positive and negative selection, and then administering them to the recipient along with MHC-specific stem cells and any desired additional donor bone marrow components, GVHD causing T-cells may be removed without fear of failure of engraftment. As a result, mixed or completely or fully allogeneic or xenogeneic repopulation can be achieved.

Typically methods of establishing an allogeneic or xenogeneic chimeric immune system comprises substantially destroying the immune system of the recipient. This may be accomplished by techniques well known to those skilled in the art. These techniques result in the substantially full ablation of the bone marrow-stem cells of the recipient. However, there may be some resistant recipient stem cells which survive and continue to produce specific immune cells. These techniques include, for example, lethally irradiating the recipient with selected levels of radiation, administering specific toxins to the recipient, administering specific monoclonal antibodies attached to toxins or radioactive isotopes, or combinations of these techniques. The present embodiment only contemplates partial conditioning of the recipient as the donor cell dose is optimized.

Bone marrow is harvested from the long bones of the donor. For allogeneic chimerism, donor and recipient are the same species; for xenogeneic chimerism, donor and recipient are different species. A cellular composition having T cell depletion is described below. A separate cellular composition comprising a high concentration of hematopoietic progenitor stem cells is separated from the remaining donor bone marrow. Separation of a cellular composition comprising a high concentration of stem cells may be accomplished by techniques such as those used to purify FC, but based on different markers, most notably CD34 stem cell separation techniques include the methods disclosed in U.S. Pat. No. 5,061,620 and the separate LC Laboratory Cell Separation System, CD34 kit manufactured by CellPro, Incorporated of Bothell, Wash. The purified donor facilitatory cell composition and purified donor stem cell composition are then preferably mixed in any ratio. However, it is not necessary to mix these cellular compositions. The key is that if donor T cells are not critical to engraftment one can find a way around them.

If the facilitatory cell is purified by negative selection using any or all of the markers disclosed herein not to be expressed on the facilitatory cell, then the resulting cellular composition will contain stem cells as well as FC and other immature progenitor cells. Antibodies directed to T cell specific markers such as anti-αβ-TCR may be used to specifically eliminate GVHD-producing cells, while retaining hematopoietic facilitatory and stem cells without a need for substantial purification. In such a case, this one cellular composition may take the place of the two cellular compositions referred to hereinabove which comprise both purified FC and purified stem cells.

The purified donor FC and purified donor stem cells are then administered to the recipient. If these cellular compositions are separate compositions, they are preferably administered simultaneously, but may be administered separately within a relatively close period of time. The mode of administration is preferably but not limited to intravenous injection.

Once administered, it is believed that the cells home to various hematopoietic cell sites in the recipient's body, including bone cavity, spleen, fetal or adult liver, and thymus. The cells become seeded at the proper sites. The cells engraft and begin establishing a chimeric immune system. Since non-universal FC must be MHC-specific, as traditionally understood, with the stem cells whose engraftment they facilitate, it is possible that both the stem cells and FC bond together to seed the appropriate site for engraftment.

The level of alloengraftment or xenoengraftment is a titratable effect which depends upon the relative numbers of syngeneic cells and allogeneic or xenogeneic cells and upon the type and degree of conditioning of the recipient. Completely allogeneic or xenogeneic chimerism should occur if the FC of the syngeneic component have been depleted by TCD procedures or other techniques, provided that a threshold number of allogeneic or xenogeneic FC are administered; and the presence of T cells to increase chimerism. A substantially equal level of syngeneic and allogeneic or xenogeneic engraftment is sought. The amount of the various cells that should be administered is calculated for a specific species of recipient. For example, in rats, the T-cell depleted bone marrow component administered is typically between about 1×10⁷ cells and 5×10⁷ cells per recipient. In mice, the T-cell depleted bone marrow component administered is typically between about 1×10⁶ cells and 5×10⁶ cells per recipient. In humans, the T-cell depleted bone marrow component administered is typically between about 1×10⁸ cells and 3×10⁸ cells per kilogram body weight of recipient. For cross-species engraftment, larger numbers of cells may be required.

In mice, the number of purified FC administered is preferably between about 1×10⁴ and 4×10⁵ FC per recipient. In rats, the number of purified FC administered is preferably between about 1×10⁶ and 30×10⁶ FC per recipient. In humans, the number of purified FC administered is preferably between about 1×10⁶ and 10×10⁶ FC per kilogram recipient.

In mice, the number of stem cells administered is preferably between about 100 and 300 stem cells per recipient. In rats, the number of stem cells administered is preferably between about 600 and 1200 stem cells per recipient. In humans, the number of stem cells administered is preferably between about 1×10⁵ and 1×10⁶ stem cells per recipient. The amount of the specific cells used will depend on many factors, including the condition of the recipient's health. In addition, co-administration of cells with various cytokines may further promote engraftment.

In addition to total body irradiation, a recipient may be conditioned by immunosuppression and cytoreduction by the same techniques as are employed in substantially destroying a recipient's immune system, including, for example, irradiation, toxins, antibodies bound to toxins or radioactive isotopes, or some combination of these techniques. However, the level or amount of agents used is substantially smaller when immunosuppressing and cytoreducing than when substantially destroying the immune system. For example, substantially destroying a recipient's remaining immune system often involves lethally irradiating the recipient with 950 rads (R) of total body irradiation (TBI). This level of radiation is fairly constant no matter the species of the recipient. Consistent xenogeneic (rat→mouse) chimerism has been achieved with 750 R TBI and consistent allogeneic (mouse) chimerism with 600R TBI. Chimerism was established by PBL typing and tolerance confirmed by mixed lymphocyte reactions (MLR) and cytotoxic lymphocyte (CTL) response.

As stated hereinbefore, the above disclosed methods may be used for establishing both allogeneic chimerism and xenogeneic chimerism. Xenogeneic chimerism may be established when the donor and recipient as recited above are different species. Xenogeneic chimerism between rats and mice, between hamsters and mice, and between chimpanzees and baboons has been established. Xenogeneic chimerism between humans and other primates is also possible. Xenogeneic chimerism between humans and other mammals is equally viable.

It will be appreciated that, though the methods disclosed above involve one recipient and one donor, the present invention encompasses methods such as those disclosed in which stem cells and purified FC from two donors are engrafted in a single recipient.

It will be appreciated that the present invention also provides methods of reestablishing a recipient's hematopoietic system by substantially destroying the recipient's immune system or immunosuppressing and cytoreducing the recipient's immune system, and then administering to the recipient syngeneic or autologous cell compositions comprising syngeneic or autologous purified FC and stem cells which are MHC-identical to the FC.

The ability to establish successful allogeneic or xenogeneic chimerism allows for vastly improved survival of transplants. The present invention provides for methods of transplanting a donor physiological component, such as, for example, organs, tissue, or cells. Examples of successful transplants in and between rats and mice using these methods include, for example, islet cells, skin, hearts, livers, thyroid glands, parathyroid glands, adrenal cortex, adrenal medullas, and thymus glands. The recipient's chimeric immune system is completely tolerant of the donor organ, tissue, or cells, but competently rejects third party grafts. Also, bone marrow transplantation confers subsequent tolerance to organ, tissue, or cellular grafts which are genetically identical or closely matched to the bone marrow previously engrafted.

Transplanted donor organ, tissue, or cells competently perform their function in the recipient. For example, transplanted islet cells function competently, and thereby provide an effective treatment for diabetes. In addition, transplantation of bone marrow using methods of the present invention can eliminate the autoimmune diabetic trait before insulin-dependence develops. Successful solid organ transplants between humans and animals may be performed using methods of the present invention involving hematopoietic FC. For example, islet cells from other species may be transplanted into humans to treat diabetes in the human recipient after the disease is diagnosed or after the onset of insulin dependence. Major organs from animal donors such as, for example, pigs, cows or fish can solve the current problem of donor shortages. For example, 50% of patients who require a heart transplant die before a donor is available. It has been demonstrated that permanent acceptance of endocrine tissue engrafts (thyroid, parathyroid, adrenal cortex, adrenal medulla, islets) occurs in xenogeneic chimeras after bone marrow transplantation from a genetically identical donor. Hence, mixed xenogeneic chimerism or fully xenogeneic chimerism established by methods of the present invention can be employed to treat endocrine disorders as well as autoimmunity, such as, for example, diabetes.

The methods of the present invention involve transplanting the specific donor physiological component by methods known to those skilled in the art and, in conjunction with establishing a chimeric immune system in the recipient using the transplant donor as the donor of the purified donor facilitatory cell composition and donor stem cell composition. A mixed chimeric immune system is preferred. The method of establishing a mixed chimeric immune system may be performed before, during, or after the transplantation, but is preferably performed before the transplantation, especially since immunosuppression and cytoreduction or immunodestruction is necessary in the chimeric methods as disclosed herein. The methods disclosed allow for both allotransplantation and xenotransplantation. Because the methods disclosed herein provide for donor-specific immunotolerance, many procedures previously necessary to resist rejection of the donor organ, tissue, or cells are unnecessary. For example, live bone and cartilage may be transplanted by the herein disclosed method.

Cell farming technology can provide for a readily available supply of FC, stem cells and genetically matched physiological donor components. For example, bone marrow cells enriched for the facilitatory cell can be propagated in vitro in cultures and/or stored for future transplantation. Cellular material from the same donor can be similarly stored for future use as grafts.

Beyond transplantation, the ability to establish a successful allogeneic or xenogeneic chimeric hematopoietic system or to reestablish a syngeneic or autologous hematopoietic system can provide cures for various other diseases or disorders which are not currently treated by bone marrow transplantation because of the morbidity and mortality associated with GHVD. Autoimmune diseases involve attack of an organ or tissue by one's own immune system. In this disease, the immune system recognizes the organ or tissue as a foreign. However, when a chimeric immune system is established, the body relearns what is foreign and what is self. Establishing a chimeric immune system as disclosed can simply halt the autoimmune attack causing the condition. Also, autoimmune attack may be halted by reestablishing the victim's immune system after immunosuppression and cytoreduction or after immunodestruction with syngeneic or autologous cell compositions as described hereinbefore. Autoimmune diseases which may be treated by this method include, for example, type I diabetes, systemic lupus erythematosus, multiple sclerosis, rheumatoid arthritis, psoriasis, colitis, and even Alzheimers disease. The use of the FC plus stem cell can significantly expand the scope of diseases which can be treated using bone marrow transplantation.

It has recently been discovered that purified hamatopoietic stem cells can differentiate into hepatocytes in vivo, see, Lagasse, E., et al., Nature Medicine, 6(11):1229-1234 (2000), which is incorporated herein by reference. In another embodiment of the present invention FC could be added to stem cells to assist in the regeneration of organs and damaged tissues, such as but not limited to heart tissue, skin, liver, lung, kidney, pancreatic tissue, organ, such as but not limited to, a thyroid gland, a parathyroid gland, a thymus, an adrenal cortex, an adrenal medulla.

Because a chimeric immune system includes hematopoietic cells from the donor immune system, deficiencies in the recipient immune system may be alleviated by a nondeficient donor immune system. Hemoglobinopathies such as sickle cell anemia, spherocytosis or thalassemia and metabolic disorders such as Hunters disease, Hurlers disease, and enzyme defects, all of which result from deficiencies in the hematopoietic system of the victim, may be cured by establishing a chimeric immune system in the victim using purified donor hematopoietic FC and donor stem cells from a normal donor. The chimeric immune system should preferably be at least 10% donor origin (allogeneic or xenogeneic).

The ability to establish successful xenogeneic chimerism can provide methods of treating or preventing pathogen-mediated disease states, including viral diseases in which species-specific resistance plays a role. For example, AIDS is caused by infection of the lymphohematopoietic system by a retrovirus (HIV). The virus infects primarily the CD4⁺ T cells and antigen presenting cells produced by the bone marrow stem cells. Some animals, such as, for example, baboons, possess native immunity or resistance to AIDS. By establishing a xenogeneic immune system in a human recipient, with a baboon or other AIDS resistant and/or immune animal as donor, the hematopoietic system of the human recipient can acquire the AIDS resistance and/or immunity of the donor animal. Other pathogen-mediated disease states may be cured or prevented by such a method using animals immune or resistant to the particular pathogen which causes the disease. Some examples include hepatitis A, B, C, and non-A, B, C hepatitis. Since the facilitatory cell plays a major role in allowing engraftment of stem cells across a species disparity, this approach will rely upon the presence of the facilitatory cell in the bone marrow inoculum.

The removal of the facilitatory cell has been shown to substantially impair engraftment across species differences. However, while not the preferred approach, untreated xenogeneic bone marrow will engraft if sufficient cells are administered. Bone marrow derived cells could be used in this case to treat or prevent AIDS with or without enrichment for the facilitatory cell. Previous studies demonstrated that GVHD could occur across a species barrier. Therefore, the preferred approach would be to establish the xenogeneic chimeric immune system using cellular compositions comprising purified donor FC by methods disclosed herein or compositions depleted of T cells.

Furthermore, some animals, such as, for example, baboons and other non-human primates, possess native immunity or resistance to hepatitis. By transplanting a liver from a baboon or other hepatitis resistant animal into a victim of hepatitis using a method of the present invention, wherein a xenogeneic chimeric immune system is established in the victim using purified donor FC plus stem cells, the donor liver will not be at risk for hepatitis, and the recipient will be tolerant of the graft, thereby eliminating the requirement for nonspecific immunosuppressive agents. Unmodified bone marrow or purified stem cells may suffice as the liver may serve as a hematopoietic tissue and may contain FC that will promote the engraftment of stem cells from the same donor.

Establishing a mixed chimeric immune system has also been found to be protective against cancer. (Sykes et al., Proc. Natl. Acad. Sci., U.S.A., 87: 5633-5637 (1990). Although the mechanism is not known, it may be due to multiplication of immune cell tumor specificity by the combination of donor and recipient immune system cells.

Usually, mixed chimerism is preferred. However, fully allogeneic or fully xenogeneic chimerism may be preferred in certain instances. For example, the present invention provides a method of treating leukemia or other malignancies of the lymphohematopoietic system comprising substantially destroying the victim's immune system and establishing a fully allogeneic chimeric immune system by the methods described herein. Since the victim's own immune system is cancerous, it is preferred to fully replace the syngeneic cells with allogeneic cells of a non-cancerous donor. In this case, autologous purified stem cells and FC may be used in order to totally eliminate all cancer cells in the donor preparation, especially if high dose chemotherapy or irradiation is used to ablate endogenous FC.

The present invention also provides methods of practicing gene therapy. It has recently been shown that sometimes even autologous cells which have been genetically modified may be rejected by a recipient. Utilizing methods of the present invention, a chimeric immune system can be established in a recipient using hematopoietic cells which have been genetically modified in the same way as genetic modification of other cells being transplanted therewith. This will render the recipient tolerant of the genetically modified cells, whether they be autologous, syngeneic, allogeneic or xenogeneic.

It will be appreciated that the present invention discloses cellular compositions comprising purified FC cellular compositions depleted of T cells with the retention of FC and stem cells, methods of purifying FC, methods of establishing fully, completely or mixed allogeneic or xenogeneic chimeric immune systems, methods of reestablishing a syngeneic immune system, and methods of utilizing compositions of FC to treat or prevent specific diseases, conditions or disorders. It will also be appreciated that the present invention discloses methods of treating or preventing certain pathogen-mediated diseases by administering xenogeneic cells which have not been purified for the facilitatory cell.

Whereas particular embodiments of the invention has been described hereinbefore, for purposes of illustration, it would be evident to those skilled in the art that numerous variations of the details may be made without departing from the invention as defined in the appended claims.

The invention is discussed in more detail in the subsections below, solely for the purpose of description and not by way of limitation. For clarity of discussion, the specific procedures and methods described herein are exemplified using a murine model; they are merely illustrative for the practice of the invention. Analogous procedures and techniques are equally applicable to all mammalian species, including human subjects.

### EXAMPLES

### Materials and Methods

Animals:

Five-to seven-week-old male ACI (RT1Aa), Wistar Furth (WF; RT1Au), and Fisher (F344; RT1A1) rats were purchased from Harlan Sprague Dawley (Indianapolis, Ind.). Animals were housed in a barrier animal facility at the Institute for Cellular Therapeutics, University of Louisville, Louisville, Ky., and cared for according to specific University of Louisville and National Institutes of Health animal care guidelines.

TCD of Bone Marrow in Vitro:

TCD was performed as described previously. Briefly, bone marrow was harvested from femurs and tibias of ACI rats by flushing with Media 199 (GIBCO, Grand Island, N.Y.) containing 2 μg/ml gentamicin (MEM), using a 22-gauge needle, and then filtered through sterile nylon mesh. Bone marrow cells were washed, counted and resuspended to 100×10⁶ cells/ml in 1×Hanks' balanced salt solution containing 10% fetal bovine serum. Cells were incubated with anti-αβ-TCR monoclonal antibody (mAb) (R73; mouse IgG1; Pharmingen, San Diego, Calif.) and/or anti-γδ-TCR mAb (V65; mouse IgG1; Pharmingen) for 30 min at 4° C. The cells were washed twice to remove unbound primary mAb and incubated for 60 minutes at 4° C. with Dynabeads M-450 (goat anti-mouse IgG) immunomagnetic beads at a bead/T cell ratio of approximately 20:1. T cells were then isolated from bone marrow by magnetic separation and the unbound bone marrow cells were removed with the supernatant. TCD-bone marrow cells were resuspended in MEM at a final concentration of 100×10⁶ cells/mL.

Verification of TCD by Blow Cytometry:

To confirm adequacy of TCD, pre-depletion cells, post-incubation cells, and post-depletion cells were incubated with anti-αβ-TCR-fluorescein isothiocyanate (FITC), anti-γδ-TCR-phycoerythrin (PE) or rat adsorbed goat antimouse Ig-FITC (Pharmingen), the secondary antibody for αβ-TCR or γδ-TCR for 30 min. The latter stain detects coating and saturation of the target cells with mAbs. These cells were also incubated with anti-CD8-FITC, anti-CD3-PE, and biotinylated anti-αβ-TCR and streptavidinconjugated antigen presenting cells (APQ (Phanningen) to enumerate CD3⁺ and CD8⁺ cell populations. Facilitating cells were enumerated using two- and three-color flow cytometry to detect CD3⁺/CD8⁺/TCR⁻ cells. Then cells were washed twice in “fluorescence-activated Cell Sorter” (FACS) medium (prepared in laboratory) and fixed in 1% paraformaldehyde (Tousimis Research Corporation, Rockville, Md.). Flow cytometric analysis was performed on a FACSCalibur (Becton Dickinson, Mountain View, Calif.).

Preparation of Mixed Allogeneic Chimeras (ACI→WF):

Mixed allogeneic chimeras were prepared by methods previously described by the inventor. Briefly, Wistur-Furth (WF) rats were conditioned with 950 cGy of TBI. Using sterile technique, recipients were reconstituted within 4-6 hours following TBI with 100×106 TCD bone marrow cells from ACI rats diluted in 1 ml MEM via penile vein injection. Control WF rats received equal numbers of untreated bone marrow cells.

Determination of Chimerism:

Thirty days post-BMT, recipients were characterized for allogeneic engraftment using two-color-flow cytometry. Chimerism was determined measuring the percentage of peripheral blood lymphocyte (PBL) of ACI or WF MHC class I antigen. Briefly, whole blood of recipients was collected in heparinized tubes, and aliquots of 100 μL were stained with purified anti-RTIAu (NR3/31; rat IgG2a; Serotec, Toronto, Ontario, Canada) and biotinylated anti-RTIAa,b (C3; LOU/cN JgG2b; Pharmingen) mAbs for 30 minutes. The cells were washed twice, then counterstained with anti-rat IgG2a -FITC (RG7/1.30; mouse IgG2b, Pharmingen) or streptavidin-conjugated (“antigen presenting cells”) APC (Pharmingen). Red blood cells were lysed with ammonium chloride lysing buffer for 5 minutes at room temperature. The cells were then washed in FACS medium and fixed in 1% paraformaldehyde.

Assessment of GVHD:

All chimeras were evaluated for manifestations of GVHD on a daily basis for the first month following reconstitution and weekly thereafter. The primary diagnosis of GVHD was based on previously described clinical criteria, which consist of diffuse erythema (particularly of the ear), hyperkeratosis of the foot pads, dermatitis, weight loss, generalized unkempt appearance, or diarrhea. An animal was considered to exhibit acute GVHD if at least four of the above signs were observed. The diagnosis of GVHD was confirmed by the histologic analysis of skin, tongue, liver, and small intestine following 30, 60, 90, 150, or 220 days. Tissues were fixed in 10% buffered formalin for routine hematoxylin and eosin (H&E) staining. Grading of GVHD was performed in a blinded fashion according to previously described histologic criteria.

Intra-Abdominal Heterotopic Cardiac Transplantation:

Four months after “bone marrow transplantation” (BMT), cardiac allografts from ACI, WF, and F344 rat donors were transplanted into mixed allogeneic chimeras as previously described. Allograft survival was assessed daily, based on the presence and quality of the graft heartbeat graded from 0 (no palpable beat) to 4 (visual pulsation).

Rejection of cardiac allografts was defined as cessation of visible or palpable cardiac contractions and was confirmed by the histologic presence of a mononuclear cell infiltrate and myocyte necrosis on H&E stained sections.

Statistical Analysis:

Experimental data were evaluated for significant differences using the Independent Sample test; P<0.05 was considered significant difference. Graft survival was calculated according to the Kaplan-Meier method.

Results

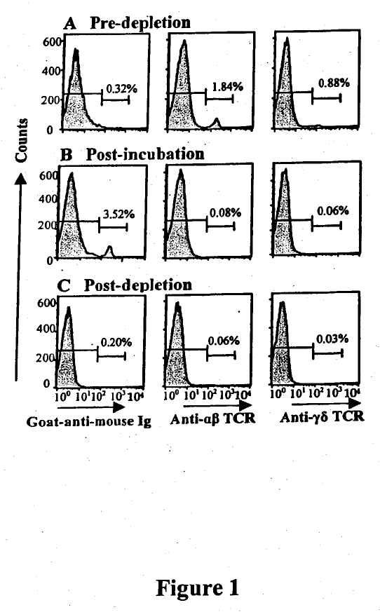

Depletion of αβ- and γδ-TCR⁺ T cells from rat marrow does not remove FC. αβ- and γδ-TCR⁺ T cells comprise 2% to 4% of the rat marrow. TCD of ACI marrow reduced the proportion of αβ-TCR⁺ T cells from 1.84%±0.99% to 0.06%±0.03%, and γδ-TCR⁺ T cells from 0.88%±0.32% to 0.03%±0.02% (Table 1). FIG. 1 illustrates T cell depletion of rat bone marrow. Adequacy of αβ- and γδ-TCR⁺ T cell depletion was confirmed using anti-αβ-TCR FITC and anti-γδ-TCR PE or rat adsorbed goat anti-mouse Ig FITC mAbs pre-depletion (A), post-incubation (B) and post-depletion (C). Staining with these mAbs demonstrated that αβ- and γδ-TCR⁺ T cells had been effectively depleted.

TABLE 1
Efficacy of T cell depletion was confirmed by flow cytometry

| | |
|---|---|
| Cells depleted | % T cell of |
| from | bone marrow (mean ± SD)^{a} |

| Donor marrow | Pre-depletion | Post-depeltion |
|---|---|---|
| αβ-TCR | 1.84 ± 0.99 | 0.06 ± 0.03 |
| γδ-TCR | 0.88 ± 0.32 | 0.03 ± 0.02 |
| αβ- and γδ-TCR | 3.40 ± 1.29 | 0.07 ± 0.01 |

^{a}Results are expressed as the mean ± SD of at least 4 experiments

Efficacy of TCD was confirmed by flow cytometry. The adequacy of depletion was further confirmed using goat anti-mouse Ig, an isotype and species-specific secondary antibody for the anti-αβ-TCR or γδ-TCR mAbs which would enumerate cells that were coated with antibody but not removed (FIG. 1, left column).

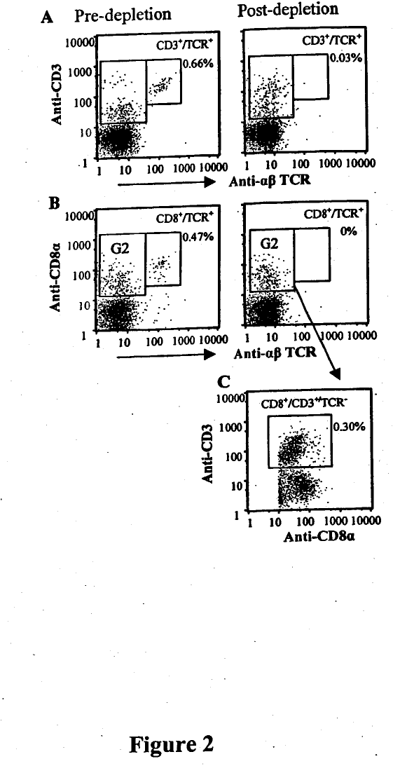

FIGS. 2A, 2B, and 2C illustrate the detection of facilitating cells. Bone marrow cells (pre- and post-depletion) were analyzed for the presence of facilitating cells using three-color-flow cytometry. Staining using anti-CD8a FITC, anti-CD3 PE and anti-αβ-TCR biotin (sandwiched with streptavidin APC mAbs showed that CD3⁺/CD8⁺/TCR⁻ cell population remains in marrow after depletion of αβ- and γδ-TCR⁺ T cells. A and B, bone marrow cells were analyzed for their CD8, CD3 and TCR expression from lymphoid gate (G1) and CD8⁺/TCR⁻ were gated (G2). C, CD8⁺/CD3⁺/TCR⁻ cells remain in marrow after TCD (from G2). A minimum of 100,000 events was counted. As indicated in FIG. 2, the FC population (CD8⁺/CD3⁺/TCR⁻) is still present in marrow after depletion of αβ-and γδ-TCR⁺ T cells at a level ranging from 0.23% to 0.45% of total cells. The CD₈^{bright} population of conventional T cells was removed while the CD8^{intermediate}/TCR⁻ FC population remained.

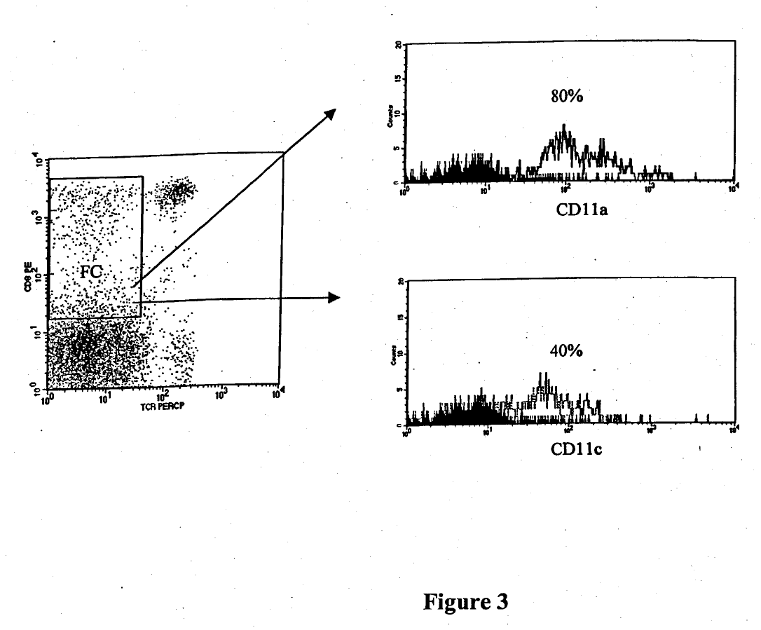

The CD8⁺/TCR⁻ FC population was also analyzed for expression of CD11a and CD11c. A minimum of 100×10³ events were analyzed. CD11a is expressed on macrophages, on monocytes, and is a developmental marker on lymphocytes. CD11b is expressed primarily on macrophages and monocytes, while CD11c is predominantly expressed on dendritic cells. Approximately 40% of the CD8⁺/TCR7 FC are CD11c⁺ (FIG. 3). Thirty-five percent of FC cells were also positive for the dendritic cell marker OX-62. CD11a was expressed on 80% of FC. The percentage of CD11a and CD11c positive cells were based on the FC gate.

Depletion of αβ- and γδ-TCR⁺ T Cells from Donor Marrow does not Impair Allogeneic Engraftment.

One hundred percent of recipient (WF) rats conditioned and transplanted with αβ and γδ-TCR⁺ T cell depleted donor marrow engrafted as chimeras (Group C). All of the recipients exhibited stable mixed HSC chimerism with 3.4% to 88.8% of total peripheral lymphoid cells of donor derivation >6 months following BMT. Seventy-five percent and eighty-six percent of recipients transplanted with either αβ-TCR⁺ T cell (Group A) or γδ-TCR⁺ T cell (Group B) depleted donor marrow engrafted. The level of donor chimerism in Group A, Group B and Group C was 73.0%±8.3%, 92.3%±9.2% and 46.3%±32.8%, respectively (Table 2).

TABLE 2
PBL typing of mixed allogeneic rat chimeras^{a}

| | | | Bone | | |
|---|---|---|---|---|---|
| | | Depletion | marrow | % Donor chimerism | |
| | | of cells from | engraft | (Mean ± SD) | |
| Group | N | bone marrow | (n %) | 30 days | 90 days |
| A | 4 | αβ-TCR | 3 (75%) | 73± | 83.5 ± 6.6 |
| B | 7 | γδ-TCR | 6 (86%) | 92.3 ± 9.2 | 94.3 ± 3.9 |
| C | 10 | αβ- and γδ-TCR | 10 (100%) | 46.3 ± 32.8^{b} | 51.1 ± 33.8 |
| D | 4 | Untreated | NA^{c} | NA | NA |

^{a}Flow cytometric analysis of lymphoid chimerism in PBL from ACI rat to WF rat.
Determination of chimerism was performed monthly 1-6 months after bone marrow transplantation. Chimerism was stable in all animals.
^{b}P < 0.05 compared to Group B; P > 0.05 compared to Group A.
^{c}Animals expired between 18 and 28 days after BMT due to severe GVHD.

Control WF rats transplanted with untreated donor ACI rat marrow expired between 18 and 28 days after BMT due to severe GVHD. Survival of recipients of αβ- and γδ-TCW T cell depleted allogeneic marrow was superior to that for chimeras that received αβ-TCR⁺ or γδ-TCR⁺ T cell depleted marrow due to avoidance of GVHD in that group.

Depletion αβ-Plus γδ-TCR⁺ T Cells from Donor Marrow is Required to Prevent GVHD.

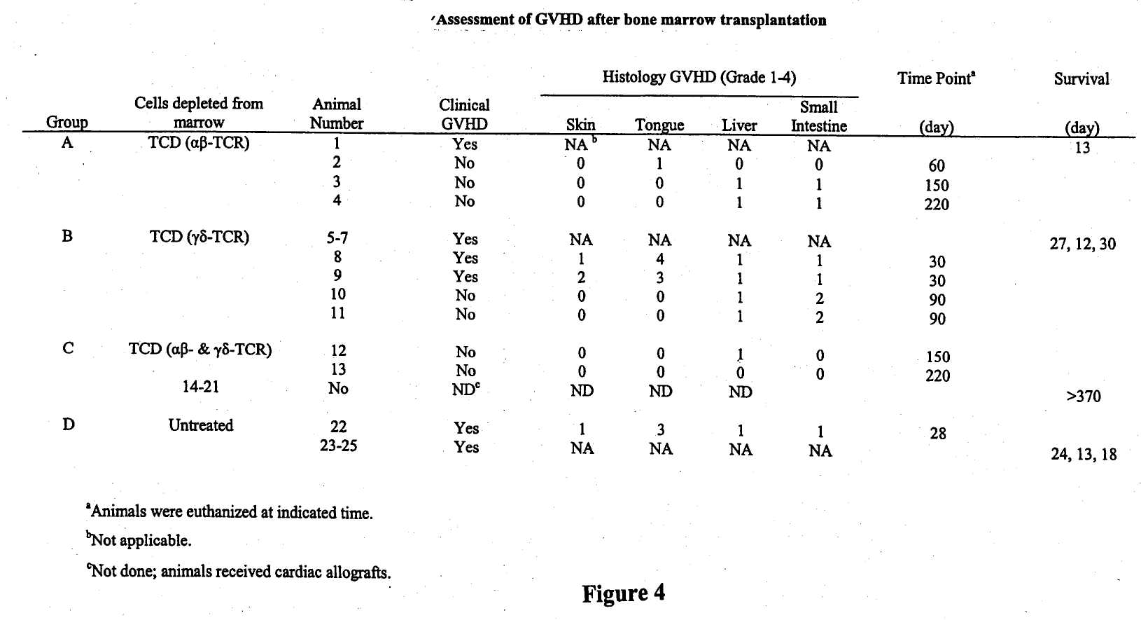

To test whether donor αβ- or γδ-TCR⁺ T cells would affect the occurrence of GVHD, chimeras were prepared with bone marrow that had been depleted of αβ-TCR⁺ (Group A), γδ-TCR⁺ (Group B), or both αβ- and γδ-TCR⁺ T cells (Group C). Recipients of untreated marrow were prepared as controls (Group D). In Group D, all four rats conditioned and reconstituted with untreated ACI bone marrow exhibited clinical signs of severe acute GVHD. Three of these animals expired before 28 days due to GVHD. Histologic examination 28 days after BMT in one rat showed severe GVHD consistent with grade 3 in tongue (FIG. 4).

Tissues from animals in Groups A, B and C were collected for histologic assessment of GVHD at 30, 60, 90, 150, and 220 days post BMT. All samples were read blind. The results are summarized in FIG. 4. In Group A, one of the 4 animals exhibited clinical signs of severe GVHD and survived to 13 days post-BMT. After 60 days post-BMT, upon histologic examination of the surviving rats, their tissues displayed mild signs of GVHD consistent with grade 1.

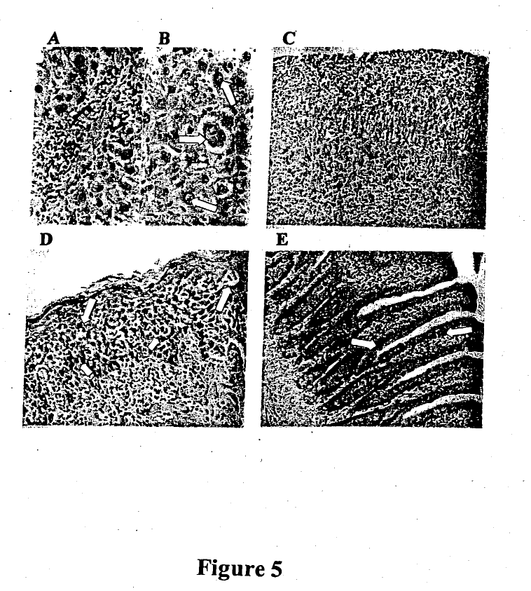

FIGS. 5A-E illustrate a histologic assessment of GVHD. Hematoxylin and eosin stained sections of skin, tongue, liver and small intestine were taken from recipient WF rats receiving 100×10⁶ TCD donor marrow depleted of αβ-TCR⁺ T cells (Group A) or γδ-TCR⁺ T cells (Group B). Liver sections from a Group A rat 150 days post-BMT showing portal and bile duct inflammation (A, original magnification×150) and apoptosis in different stages of development (B, arrows, original magnification×150). Tongue from a Group B rat 30 days post BMT exhibiting severe inflammation and necrosis of mucosa which is totally denuded. The underlying muscle layer was also inflamed. Granulation tissue with numerous capillaries was also present (C, original magnification×200). The skin from a Group B rat 30 days post-BMT showing moderate mononuclear cell infiltrate in the epidermis as well in dermal layer. Clusters of prominent lymphocytes replace the keratinocytes in the epidermis (arrows). Apoptotic bodies (short arrows) are frequently observed (D, original magnification×150). Small intestine from a Group B rat 90 days post-BMT with evidence of lymphocyte infiltration in the mucosal cells with apoptosis also present (arrows). Regeneration of crypts with mitosis is also noted (E, original magnification×150). The liver revealed mild focal mononuclear cell infiltrate within the portal tracts and in the periductal areas and regenerative change with spotty liver cell necrosis (FIGS. 5A and 5B). Examination of the intestine revealed very mild lymphocytic ileitis with crypt hyperplasia. These data therefore confirm that γδ-TCR⁺ T cells alone are sufficient to mediate GVHD.

In Group B, 5 of 7 rats exhibited clinical signs of GVHD. Three of the rats died 30 days post-BMT. The remaining two rats showed histological moderate to severe signs of GVHD and necrosis consistent with grade 3 to 4 by 30 days post BMT. The tongue revealed severe inflammation and necrosis (FIG. 5C). The skin revealed moderate mononuclear cell infiltrate in the epidermis which showed rare apoptotic bodies (FIG. 5D). The liver showed mild cholangitis and mild liver cell necrosis. The intestine showed mild lymphocytic ileitis. Ninety days post-BMT, the two rats which showed no clinical signs of GVHD revealed moderate lymphocytic ileitis on histology (FIG. 5E). These data therefore confirm that αβ-TCR⁺ T cells are the primary effector cells for severe acute GVHD.

None of the animals in which the marrow had been depleted of αβ- and γδ-TCR⁺ T cells showed clinical signs of GVHD (Group C). However, one rat did have rare lymphocytes within the bile duct epithelium in the liver 150 days post-BMT. One rat displayed no histological evidence of GVHD 220 days post-BMT. These data therefore suggest that both αβ- and γδ-TCR⁺ T cells mediate clinically significant GVHD, although the severity of GVHD differs. If αβ-TCR⁺ T cells remain in the marrow inoculum, GVHD is more severe and more frequent compared with γδ-TCR⁺ T cells.

Evidence for Tolerance to Donor-Specific Cardiac Allografts.

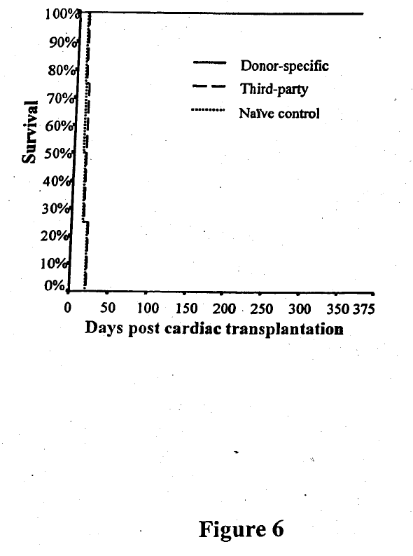

To test whether mixed chimerism achieved with transplantation of marrow depleted of both αβ- and γδ-TCR⁺ T cells would induce donor-specific tolerance, heterotopic cardiac grafts from ACI (marrow donor) or F344 (third-party) rats were performed. FIG. 6 illustrates the survival of heterotopic cardiac allografts in mixed allogeneic chimeras (ACI→WF). Donor-specific (ACI) or third-party (F344) cardiac grafts were transplanted 4 months after BMT. ACI hearts were transplanted to naive WF rats as controls. Graft survival was determined by palpation and rejection confirmed by pathology. Survival of donor-specific grafts was significantly greater than for third party and controls. As shown in FIG. 6, donor-specific cardiac allografts were permanently accepted by mixed allogeneic chimeras (MST≧375 days), whereas third party (F344) grafts were promptly rejected (MST=15 days). Upon histological examination, all the nonfunctioning grafts had evidence of myocyte necrosis and mononuclear cell infiltration consistent with acute rejection. In contrast, donor-specific allografts showed no evidence for myocytolysis or cellular infiltration. Moreover, there was no evidence for chronic rejection (FIG. 6).

The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof.

The foregoing description is considered as illustrative only of the principles of the invention. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims which follow.

The embodiments of the invention in which an exclusive property or privilege is claimed are defined as follows:

1. A cellular composition comprising mammalian hematopoietic cells, which are depleted of graft-versus-host-disease-producing cells having a phenotype of αβTCR⁺, with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells.

2. A cellular composition comprising human hematopoietic cells, which are depleted of graft-versus-host-disease-producing cells having a phenotype of αβTCR⁺, with the retention of mammalian hematopoietic facilitatory cells having a phenotype of CD8⁺/TCR⁺, CD8⁺/TCR⁻, which hematopoietic facilitatory cells are capable of facilitating engraftment of bone marrow cells.

3. A method of partially or completely reconstituting a mammal's lymphohematopoietic system comprising administering to the mammal the cellular composition of claim 2.

4. The method of claim 3 in which the mammal is conditioned by total body irradiation.

5. The method of claim 3 in which the mammal is conditioned by an immunosuppressive agent.

6. The method of claim 3 in which the mammal is conditioned by a cytoreduction agent.

7. The method of claim 3 in which the pharmaceutical composition is administered intravenously.

8. The method of claim 3 in which the mammal is a human.

9. The method of claim 3 in which the mammal suffers from autoimmunity.

10. The method of claim 9 in which the autoimmunity is diabetes.

11. The method of claim 9 in which the autoimmunity is multiple sclerosis.

12. The method of claim 9 in which the autoimmunity is systemic lupus erythematosus.

13. The method of claim 3 in which the mammal suffers from immunodeficiency.

14. The method of claim 3 in which the mammal is infected with a human immunodeficiency virus.

15. The method of claim 3 in which the mammal is infected with a hepatitis virus.

16. The method of claim 3 in which the mammal suffers from a hematopoietic malignancy.

17. The method of claim 3 in which the mammal suffers from anemia.

18. The method of claim 3 in which the mammal suffers from hemoglobinopathies.

19. The method of claim 3 in which the mammal suffers from an enzyme deficiency state.

20. The method of claim 3 in which the mammal is human and the cellular composition is obtained from a human.

21. The method of claim 3 in which the mammal is human and the pharmaceutical composition is obtained from a non-human animal.

22. A method of inducing tissue or organ regeneration in a mammal comprising administering to the mammal FC plus HSC cells.

23. The method of claim 22 in which the donor organ is heart.

24. The method of claim 22 in which the donor organ is skin.

25. The method of claim 22 in which the donor organ is liver.

26. The method of claim 22 in which the donor organ is lung.

27. The method of claim 22 in which the donor organs are heart and lung.

28. The method of claim 22 in which the donor organ is kidney.

29. The method of claim 22 in which the donor tissues are pancreatic islet cells or whole pancreas.

30. The method of claim 21 in which the donor organ is an endocrine organ.

31. The method of claim 30 in which the endocrine organ is a thyroid gland.

32. The method of claim 30 in which the endocrine organ is a parathyroid gland.

33. The method of claim 30 in which the endocrine organ is a thymus.

34. The method of claim 30 in which the endocrine organ is adrenal cortex.

35. The method of claim 30 in which the endocrine organ is adrenal medulla.

36. The method of claim 22 in which the donor cells are neurons.

37. The method of claim 22 in which the donor cells are myocytes.
